(19) United States
(12) Patent Application Publication
(10) Pub. No.: US 2021/0121475 A1
(43) Pub. Date: Apr. 29, 2021

(54) IMIPRAMINE COMPOSITIONS AND METHODS OF TREATING CANCER

(71) Applicant: THE BOARD OF REGENTS OF THE UNIVERSY OF TEXAS SYSTEM, Austin, TX (US)

(72) Inventors: Manjeet Rao, San Antonio, TX (US); Subapriya Rajamanickam, San Antonio, TX (US); Santosh Timilsina, San Antonio, TX (US); Virginia G. Kaklamani, San Antonio, TX (US)

(21) Appl. No.: 16/617,414

(22) PCT Filed: Jun. 20, 2018
(86) PCT No.: PCT/US2018/038436

§ 371 Date: Nov. 26, 2019

Related U.S. Application Data
(60) Provisional application No. 62/522,203, filed on Jun. 20, 2017.

(51) Int. Cl. A61K 31/55; A61K 31/502; A61K 9/107; A61P 35/00; A61K 31/454; A61K 39/395; A61P 35/04

(52) U.S. Cl. CPC A61K 31/55; A61K 31/502; A61K 9/107; A61P 35/04; A61K 31/454; A61K 39/3955; A61P 35/00

(57) ABSTRACT

The disclosure relates to compositions and methods of treating cancer in a subject. The method comprises administering to a patient in need of treatment an effective amount of imipramine.

### CROSS REFERENCE TO RELATED APPLICATIONS

This application claims the benefit of U.S. Provisional Application No. 62/522,203, filed on Jun. 20, 2017. The entire contents of which are hereby incorporated by reference.

### BACKGROUND

Triple-negative breast cancer (TNBC) is an aggressive malignancy with a poor prognosis despite initial response to chemotherapy (Amos et al.). TNBCs occur more frequently in younger patients (<50 years old) and progresses aggressively with a tendency to relapse as distant metastases (Gluz et al., 2009). Despite a better chemotherapy response rate in early-stages, more than 60% of patients with TNBCs develop drug resistance leading to early relapse and shorter survival (Gluz et al., 2009). Therefore, a more robust approach to target TNBC with novel therapies is needed.

### SUMMARY

Disclosed herein are methods of treating cancer in a subject, the method comprising: identifying a subject in need of treatment; and administering to the subject a therapeutically effective amount of imipramine.

Disclosed herein are methods of treating cancer in a subject, the method comprising: administering a therapeutically effective amount of imipramine to a subject in need thereof.

Disclosed herein are methods of treating cancer in a subject, the method comprising: administering a therapeutically effective amount of imipramine and a PARP inhibitor to a subject in need thereof.

Disclosed herein are methods of treating cancer in a subject, the method comprising: administering a therapeutically effective amount of imipramine and a PD-L1 inhibitor to a subject in need thereof.

Disclosed herein are methods of treating cancer in a subject, the method comprising: identifying a subject in need of treatment; and administering to the subject a therapeutically effective amount of imipramine and a PARP inhibitor.

Disclosed herein are methods of treating cancer in a subject, the method comprising: identifying a subject in need of treatment; and administering to the subject a therapeutically effective amount of imipramine and a PD-L1 inhibitor.

Disclosed herein are methods of treating cancer in a subject, the method comprising: identifying a subject in need of treatment; and administering to the subject a therapeutically effective amount of imipramine and a PD-1 inhibitor.

Disclosed herein are methods of inhibiting cell cycle progression, cell growth or DNA repair, the method comprising: contacting a cell or tissue or administering to a subject in need thereof, a therapeutically effective amount of imipramine.

Disclosed herein are methods of inhibiting growth, transformation or metastasis of cancer cells, the method comprising: contacting a cell or tissue or administering to a subject in need thereof, a therapeutically effective amount of imipramine.

Disclosed herein are pharmaceutical compositions comprising: imipramine; and a PARP inhibitor, a PD-L1 inhibitor or a PD-1 inhibitor; and optionally, a pharmaceutical acceptable carrier; wherein imipramine, the PARP inhibitor, the PD-L1 inhibitor and a PD-1 inhibitor are present in a therapeutically effective amount.

Other features and advantages of the present compositions and methods are illustrated in the description below, the drawings, and the claims.

### BRIEF DESCRIPTION OF THE DRAWINGS

FIG. 1 shows that imipramine inhibits the growth of breast cancer cells in a dose- and time-dependent manner. MDA-MB-231 and MDA-MB-468 cells were treated with vehicle control (DMSO) or indicated doses of imipramine (5-100 μM) for 96 hours. The data shown are mean±SEM. *, P<0.05; **, P<0.01; ***, P<0.001; ****, P<0.0001 versus control group, t-test.

FIGS. 2A-B show that imipramine inhibits the migration and invasion of breast cancer cells. Photomicrographs of migrated (FIG. 2A) and invaded (FIG. 2B) MDA-MB-231 cells pre-treated with vehicle or imipramine (20 μM for 48 hrs) are shown.

FIGS. 3A-C show that imipramine inhibits breast cancer growth in vivo. FIG. 3A is a line graph showing mean tumor volume for vehicle or imipramine (n=7) treated mice. FIG. 3B shows photographs of tumors in control and imipramine-treated groups. FIG. 3C shows that imipramine treatment reduced tumor weight in a dose dependent manner. *, P<0.01; **, P<0.001; ***, P<0.0001 versus control group, t-test.

FIGS. 4A-B show that combination of imipramine+olaparib inhibits breast cancer growth in vivo. FIG. 4A is a line graph showing mean tumor volume for vehicle, imipramine, olaparib or imipramine+olaparib treated mice (n=7). FIG. 4B shows photographs of tumors of control (vehicle), imipramine, olaparib, and imipramine+olaparib-treated groups. **, P<0.01; ***, P<0.001 versus control group, t test.

FIGS. 5A-B show that imipramine inhibits genes associated with DNA repair. FIG. 5A. shows western blot analysis of MDA-MB-231 and MDA-MB-468 cells treated with vehicle or imipramine (40 μM) using antibodies against indicated proteins. FIG. 5B shows western blot analysis of MDA-MB 231 cells treated with either vehicle or imipramine (20 and 40 μM) for 96 hours using antibodies against indicated proteins. Membranes were reprobed with different antibodies and with (3-actin, which served as a loading control. Blots shown are representative of at least two independent experiments.

FIG. 6 is a schema showing details of phase I clinical trials with imipramine alone and in combination with niraparib/anti-PD-1 antibody.

FIG. 7 shows that imipramine inhibits PD-L1 expression. Flow cytometry analysis of cell surface PD-L1 expression in MDA-MB-231 cells treated with different doses of imipramine (10, 20 and 40 μM) using FITC-anti-PD-L1 antibody.

FIG. 8 shows that imipramine inhibits PD-L1 expression. Flow cytometry analysis of cell surface PD-L1 expression in MDA-MB-231 cells treated with imipramine (20 μM) or FOXM1-siRNA using FITC-anti-PD-L1 antibody.

FIG. 9 shows IPA analysis showing highly altered pathways in imipramine-treated MDA-MB-231 cell (left). Western blot of TNBC cells treated with vehicle or imipramine for 96 hours using antibodies against indicated proteins (right). Membranes were re-probed with (3-actin/tubulin.

### DETAILED DESCRIPTION

The present disclosure can be understood more readily by reference to the following detailed description of the invention, the figures and the examples included herein.

Before the present compositions and methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

As used in the specification and the appended claims, the singular forms “a,” “an” and “the” include plural referents unless the context clearly dictates otherwise.

The word “or” as used herein means any one member of a particular list and also includes any combination of members of that list.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “subject” refers to the target of administration, e.g., a human. Thus, the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, the subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been diagnosed with a need for treatment for cancer, such as, for example, prior to the administering step.

As used herein, the term “treating” refers to partially or completely alleviating, ameliorating, relieving, delaying onset of, inhibiting or slowing progression of, reducing severity of, and/or reducing incidence of one or more symptoms or features of a particular disease, disorder, and/or condition. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. For example, the disease, disorder, and/or condition can be cancer.

As used herein, the term “inhibit” or “inhibiting” mean decreasing tumor cell growth rate from the rate that would occur without treatment and/or causing tumor mass (e.g., cancer) to decrease. Inhibiting also include causing a complete regression of the tumor (e.g., cancer).

Defects in the DNA damage response (DDR) are a major factor predisposing normal cells to acquire oncogenic mutations. This predisposition is mainly because despite defective DDR, cancer cells continue DNA replication that results in genomic instability and consequently cancer growth and progression. Furthermore, after tumors develop, the ability of cancer cells to repair chemotherapy-induced DNA damage is one of the mechanisms for therapy resistance. Importantly, impaired DDR, though makes tumors highly immunogenic, may also facilitate escape of tumor cells from immunosurveillance via either increased production of immunosuppressive cytokines (e.g. IL-10) (Nishigori et al., 1996; Yarosh et al., 2002), or regulatory regulatory T cells (Curiel et al., 2004) or other factors in the tumor microenvironment and/or exhaustion of tumor infiltrating T cells. Therapeutics that block cell cycle progression and suppress DNA repair responses of cancer cells would be of great interest.

Described herein are results showing that the anti-depressant imipramine inhibits cell cycle progression and DNA repair by inhibiting genes such as cyclin D1, PLK1 and Rad51 in triple negative breast cancer (TNBC) cells. This DNA repair effect is significant given that the DNA repair pathway was identified as one of the most deregulated pathways in TNBC compared to other non-TNBC and benign tumors (Albiges et al., 2014). The Examples herein show that systemic delivery of imipramine suppressed TNBC growth without inducing toxicity in pre-clinical mouse models. Furthermore, the data described herein demonstrates that imipramine improves the efficacy of the poly ADP ribose polymerase (PARP) inhibitor olaparib in TNBC. Olaparib blocks repair of damaged DNA and is currently in clinical trials for treating BRCA1-mutated cancers. Imipramine inhibits the expression of the immunosuppressive cytokine IL-10 and elevates IL-12, which then activates innate immunity via natural killer (NK) cells and adaptive immunity via cytotoxic T-lymphocytes (Mukherjee et al., 2014). IL-10 is induced by programmed death-1 (PD-1) (Said et al., 2010) and programmed death ligand-1 PD-L1 (Curiel et al., 2003) and PD-1 is known to act together with IL-10 to inhibit the activity of tumor-specific CD8+ T cells (Sun et al., 2015). The PD-1 and/or PD-L1 pathway is a target for FDA approved immunotherapy against several types of cancers (Hamid et al., 2013).

Described herein are studies regarding imipramine and whether imipramine 1) is an effective drug for treating TNBC, 2) can enhance PARP inhibitor response by targeting DNA damage response, and 3) can improve immuno-surveillance and anti-PD-L1 and/or PD-1 immunotherapy through multiple mechanisms. Imipramine was first tested for its ability to be a potent therapeutic for treating TNBC. As imipramine can cross the blood-brain barrier, the studies described herein can test the utility of imipramine for treating breast cancer brain metastasis in a pre-clinical model. Next, tissues and blood collected from a pre-clinical cell line/animal model system can be carried out to confirm that imipramine acts by targeting the DNA damage response as well as by targeting the PD-1 and/or PD-L1 immunomodulatory axis. This pre-clinical cell line/animal model system can also be used to address whether imipramine's effect on DDR can alter the level/function of immunosuppressive and inflammatory cytokines, NK cells (such as shedding of receptor/ligand on NK cells) and regulatory T-cell to improve immuno-surveillance and efficacy of anti-PD-L1. Lastly, it can be tested whether imipramine may affect expression/function of DNA repair genes to improve niraparib response for inhibiting breast cancer brain metastasis.

Targeting DNA damage response in TNBC. The term DDR involves several events including loss of DDR pathway protein/s, increased replication stress, increased DNA damage and DNA repair ability of cancer cells that play an important role in facilitating tumor growth and progression. Moreover, the highly impaired DDR resulting in increased genomic instability can be a source of neoantigens in TNBC, which in theory makes TNBC an immunogenic cancer and a candidate for immunotherapy (Stephens et al., 2012). Indeed, increased tumor infiltrating leukocytes (TILs) have been reported in TNBCs (Garcia-Teijido et al., 2016). However, it is also true that despite increasing TIL, TNBC still progress. This may be due to the presence of immunosuppressive factors such as cytokines (for example, IL-10) in tumor microenvironment and/or exhaustion of tumor infiltrating T cells (Stagg and Allard, 2013). Therefore, drugs that can target DDR and inhibit immunosuppressive cytokines in tumor microenvironment can have a favorable outcome.

Imipramine is a potent therapeutic regimen. Imipramine is a tricyclic antidepressant (TCA) used in the treatment of major depressive disorder. It achieves its effects by inhibiting serotonin re-uptake and norepinephrine. It is approved by the FDA for the treatment of depression and childhood enuresis. Disclosed herein is evidence that imipramine inhibits TNBC growth by affecting DDR. Supporting evidence also suggests that imipramine can inhibit TNBC growth by inhibiting immunosuppressive environment. Also disclosed herein is evidence imipramine can improve the efficacy of other cancer treatment drugs.

Methods of Treatment

Disclosed herein, are methods of treating cancer in a subject, the method comprising: (a) identifying a subject in need of treatment; and (b) administering to the subject a therapeutically effective amount of imipramine. Also, disclosed herein, are methods of treating cancer in a subject, the method comprising: (a) identifying a subject in need of treatment; and (b) administering to the subject a therapeutically effective amount of imipramine and a therapeutically effective amount of a PARP inhibitor. In an aspect, methods of treating cancer in a subject, described herein: further comprises a therapeutically effective amount of a PD-L1 inhibitor. Also, disclosed herein, are methods of treating cancer in a subject, the method comprising: (a) identifying a subject in need of treatment; and (b) administering to the subject a therapeutically effective amount of imipramine and a therapeutically effective amount of a PD-L1 inhibitor. Also, disclosed herein, are methods of treating cancer in a subject, the method comprising: (a) identifying a subject in need of treatment; and (b) administering to the subject a therapeutically effective amount of imipramine and a therapeutically effective amount of a PD-1 inhibitor. Also, disclosed herein, are methods of treating cancer in a subject, the method comprising: (a) identifying a subject in need of treatment; and (b) administering to the subject a therapeutically effective amount of imipramine and a therapeutically effective amount of a PD-L1 inhibitor and a therapeutically effective amount of a PD-1 inhibitor. Also, disclosed herein, are methods of treating cancer in a subject, the method comprising: (a) identifying a subject in need of treatment; and (b) administering to the subject a therapeutically effective amount of imipramine and a therapeutically effective amount of a PD-L1 inhibitor, a therapeutically effective amount of a PD-1 inhibitor and a therapeutically effective amount of a PARP inhibitor. In an aspect, PARP inhibitor is olaparib, the PD-L1 inhibitor is an anti-PD-L1 antibody and the PD-1 inhibitor is an anti-PD-1 antibody.

Disclosed herein are methods of inhibiting cell cycle progression, cell growth or DNA repair. The method can include contacting a cell or tissue or administering to a subject in need thereof, a therapeutically effective amount of imipramine. In an aspect, the method further comprises administering a therapeutically effective amount of a PARP inhibitor. In an aspect, the administration of imipramine increases the efficacy of olaparib. In an aspect, the method further comprises administering a therapeutically effective amount of a PD-L1 inhibitor. In an aspect, the method further comprises administering a therapeutically effective amount of a PD-1 inhibitor. In an aspect, the method further comprises administering a therapeutically effective amount of a PARP inhibitor; and a PD-L1 inhibitor or a PD-1 inhibitor. In an aspect, the method comprises administering olaparib; niraparib, veliparib or talazoparib; a PD-L1 antibody; and an anti PD-1 antibody. In some aspects, the method includes inhibiting cell cycle progression, cell growth or DNA repair by inhibiting one or more of genes cyclin D1, PLK1 or Rad51.

The cell cycle, or cell-division cycle, is the series of events that take place in a cell leading to its division and replication. The cell cycle consists of four distinct phases. Activation of each phase is dependent on the proper progression and completion of the previous one. Cells that have temporarily or reversibly stopped dividing are said to have entered a state of quiescence. Each phase of the cell cycle has a distinct set of specialized biochemical processes that prepare the cell for initiation of cell division.

Disclosed herein are methods of inhibiting growth, transformation or metastasis of cancer cells. In an aspect, the cancer cells can be mammalian cells. The method can include contacting a cell or tissue or administering to a subject in need thereof, a therapeutically effective amount of imipramine. In an aspect, the method further comprises administering a therapeutically effective amount of a PARP inhibitor. In an aspect, the administration of imipramine increases the efficacy of olaparib. In an aspect, the method further comprises administering a therapeutically effective amount of a PD-L1 inhibitor. In an aspect, the method further comprises administering a therapeutically effective amount of a PD-1 inhibitor. In an aspect, the method further comprises administering a therapeutically effective amount of a PARP inhibitor; and a PD-L1 inhibitor or a PD-1 inhibitor. In an aspect, the method comprises administering olaparib; niraparib, veliparib or talazoparib; a PD-L1 antibody; and an anti PD-1 antibody.

In an aspect, the PARP inhibitor is olaparib, niraparib, veliparib or talazoparib. In an aspect, the PARP inhibitor is olaparib.

In an aspect, the PD-L1 inhibitor is anti-PD-L1 antibody. In an aspect, the anti-PD-L1 antibody is BMS-936559, durvalumab, atezolizumab or avelumab.

In an aspect, PD-1 inhibitor is an anti-PD-1 antibody. In an aspect, the anti-PD-1 antibody is nivolumab, pembrolizumab or TSR-042.

The compositions described herein can be formulated to include a therapeutically effective amount of imipramine and one or more of the compounds (e.g., PARP inhibitor, PD-L1 inhibitor and/or PD-1 inhibitor) described herein. Therapeutic administration encompasses prophylactic applications. Based on genetic testing and other prognostic methods, a physician in consultation with their patient can choose a prophylactic administration where the patient has a clinically determined predisposition or increased susceptibility (in some cases, a greatly increased susceptibility) to a type of cancer.

The compositions described herein can be formulation in a variety of combinations. The particular combination of imipramine with one or more of a PARP inhibitor, a PD-L1-inhibitor and a PD-1 inhibitor can vary according to many factors, for example, the particular the type and severity of the cancer.

The compositions described herein can be administered to the subject (e.g., a human patient) in an amount sufficient to delay, reduce, or preferably prevent the onset of clinical disease. Accordingly, in some aspects, the patient is a human patient. In therapeutic applications, compositions are administered to a subject (e.g., a human patient) already with or diagnosed with cancer in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of a composition (e.g., a pharmaceutical composition) can be an amount that achieves a cure, but that outcome is only one among several that can be achieved. As noted, a therapeutically effective amount includes amounts that provide a treatment in which the onset or progression of the cancer is delayed, hindered, or prevented, or the cancer or a symptom of the cancer is ameliorated. One or more of the symptoms can be less severe. Recovery can be accelerated in an individual who has been treated.

In some aspects, the cancer is a primary or secondary tumor. In an aspect, the cancer is a metastatic tumor. In other aspects, the primary or secondary tumor is within the patient's breast, lung, lung or liver. In yet other aspects, the cancer has metastasized. In some aspects, the cancer may originate in the breast and metastasize to one or more of the following sites: the breast, lung, liver or bone.

Disclosed herein, are methods of treating a patient with cancer. The cancer can be any cancer. In some aspects, the cancer is breast cancer, lung cancer, brain cancer or liver cancer. In an aspect, the subject has been diagnosed with cancer prior to the administering step. In an aspect, the cancer is triple negative breast cancer.

The compositions described herein can be formulated to include a therapeutically effective amount of imipramine alone or in combination with one or more of the compounds disclosed herein (e.g., PARP inhibitor, PD-L1 inhibitor and/or PD-1 inhibitor). In an aspect, imipramine can be contained within a pharmaceutical formulation. In an aspect, the pharmaceutical formulation can be a unit dosage formulation.

The therapeutically effective amount or dosage of the imipramine, any of the PARP inhibitors, PD-L1 inhibitors and PD-1 inhibitors used in the methods as disclosed herein applied to mammals (e.g., humans) can be determined by one of ordinary skill in the art with consideration of individual differences in age, weight, sex, other drugs administered and the judgment of the attending clinician. Variations in the needed dosage may be expected. Variations in dosage levels can be adjusted using standard empirical routes for optimization. The particular dosage of a pharmaceutical composition to be administered to the patient will depend on a variety of considerations (e.g., the severity of the cancer symptoms), the age and physical characteristics of the subject and other considerations known to those of ordinary skill in the art. Dosages can be established using clinical approaches known to one of ordinary skill in the art.

The duration of treatment with any composition provided herein can be any length of time from as short as one day to as long as the life span of the host (e.g., many years). For example, the compositions can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present compositions can be administered once (or twice, three times, etc.) daily, weekly, monthly, or yearly.

Dosages of imipramine can be in the range of 75 mg to 100 to 300 mg/day. In an aspect, the dosage of imipramine can be 25, 50, 75, 100, 200, or 300 mg total or any amount in between. In an aspect, the therapeutically effective dose of imipramine may be less when combined with one or more of the compounds disclosed herein. In an aspect, the administration of imipramine increases the efficacy of a PARP inhibitor. In an aspect, the administration of imipramine increases the efficacy of olaparib. Dosages of olaparib can be in the range of 100 mg to 400 mg/day or any amount in between.

Dosages of olaparib can be in the range of 100 mg to 400 mg/day. In an aspect, the dosage of olaparib can be 100, 200, 300 or 400 mg total or any amount in between.

Dosages of niraparib can be in the range of 100 mg to 300 mg/day. In an aspect, the dosage of niraparib can be 100, 200, or 300 mg total or any amount in between.

Dosages of veliparib can be in the range of 50 mg to 400 mg/day. In an aspect, the dosage of veliparib can be 50, 75, 100, 200, 300 or 400 mg total or any amount in between.

Dosages of talazoparib can be in the range of 0.6 mg to 1 mg/day. In an aspect, the dosage of talazoparib can be 0.6, 0.7, 0.8, 0.9, or 1 mg total or any amount in between.

Dosages of pembrolizumab can be in the range of 2-10 mg/kg body weight to 200 mg/day every three weeks. In an aspect, the dosage of nivolumab can be 2, 3, 4, 5, 6, 7, 8, 9 or 10 mg or any amount in between.

Dosages of TSR-042 can be in the range of 0.3-10 mg/kg body weight once every two weeks. In an aspect, the dosage of TSR-042 can be 0.3, 0.4, 0.5, 0.6, 0.7, 0.8, 0.9, 1, 2, 3, 4, 5, 6, 7, 8, 9 or 10 mg/kg or any amount in between.

Dosages of nivolumab can be in the range of 3 mg/kg body weight to 240 mg/day twice a week.

Dosages of durvalumab can be in the range of 20 mg/kg body weight every four weeks.

Dosages of atezolizumab can be in the range of 1200 mg body every three weeks.

Dosages of BMS-936559 can be in the range of 0.3-10 mg/kg every two weeks. In an aspect, the dosage of BMS-936559 can be 0.3, 0.4, 0.5, 0.6, 0.7, 0.8, 0.9, 1, 2, 3, 4, 5, 6, 7, 8, 9 or 10 mg/kg or any amount in between.

Suitable treatment regimens using any of the dosages described herein include, but are not limited to: imipramine, daily once; olaparib, twice a day; niraparib, daily once; veliparib, twice a day; talazoparib, once a day; atezolizumab, every 3 weeks; durvalumab, every 4 weeks; BMS-936559, every two weeks; nivolumab, every 2 weeks; pembrolizumab, every 3 weeks; imipramine+olaparib, imipramine, daily once+olaparib, daily twice; imipramine+niraparib, imipramine daily once+niraparib once daily; vmipramine+veliparib, imipramine daily once+veliparib daily twice; imipramine+talazoparib, imipramine daily once+talazoparib daily once; imipramine+atezolizumab, imipramine daily once+atezolizumab every 3 weeks; imipramine+durvalumab, imipramine daily once+durvalumab every 4 weeks; imipramine+nivolumab, imipramine daily once+nivolumab every 2 weeks; imipramine+pembrolizumab, imipramine daily once+pembrolizumab every 3 weeks; imipramine+BMS-936559, imipramine daily once+BMS-936559 every 2 weeks; imipramine+TSR-042, imipramine daily once+TSR-042 once every two weeks.

The total effective amount of the compositions as disclosed herein can be administered to a subject as a single dose, either as a bolus or by infusion over a relatively short period of time, or can be administered using a fractionated treatment protocol in which multiple doses are administered over a more prolonged period of time. Alternatively, continuous intravenous infusions sufficient to maintain therapeutically effective concentrations in the blood are also within the scope of the present disclosure.

The compositions described herein can be administered in conjunction with other therapeutic modalities to a subject in need of therapy. The present compounds can be given to prior to, simultaneously with or after treatment with other agents or regimes. For example, imipramine alone or with any of the compounds disclosed herein can be administered in conjunction with standard therapies used to treat cancer. In an aspect, any of the compounds or compositions described herein can be administered or used together with chemotherapy. In an aspect, imipramine and the PARP inhibitor are co-formulated. In an aspect, imipramine, the PARP inhibitor and the PD-L1 inhibitor are co-formulated. In an aspect imipramine, the PARP inhibitor, the PD-L1 inhibitor and the PD-1 inhibitor are co-formulated.

Any of the compounds or compositions described herein can be administered as a term “combination.” It is to be understood that, for example, imipramine can be provided to the subject in need, either prior to administration of a PARP inhibitor, a PD-L1 inhibitor and/or a PD-1 inhibitor or any combination thereof, concomitant with administration of said PARP inhibitor, a PD-L1 inhibitor and/or a PD-1 inhibitor or any combination thereof (co-administration) or shortly thereafter.

In an aspect, cancer cells can be sensitized prior to the administration of a PARP inhibitor, a PD-L1 inhibitor and/or a PD-1 inhibitor or any combination thereof comprising administering to a subject in need an amount (e.g., a therapeutic amount) of a PARP inhibitor, a PD-L1 inhibitor and/or a PD-1 inhibitor in combination with an amount (e.g., a sensitizing amount; or an amount that is less than what is typically recommended) of imipramine.

Pharmaceutical Compositions

As disclosed herein, are pharmaceutical compositions, comprising one or more of the therapeutic compositions or inhibitors disclosed herein. As disclosed herein, are pharmaceutical compositions, comprising imipramine and a pharmaceutical acceptable carrier described herein. In some aspects, imipramine can be formulated for oral or parental administration. In an aspect, the parental administration is intravenous, subcutaneous, intramuscular or direct injection. The compositions can be formulated for administration by any of a variety of routes of administration, and can include one or more physiologically acceptable excipients, which can vary depending on the route of administration. As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed.

The compositions can be administered directly to a subject. Generally, the compositions can be suspended in a pharmaceutically acceptable carrier (e.g., physiological saline or a buffered saline solution) to facilitate their delivery. Encapsulation of the compositions in a suitable delivery vehicle (e.g., polymeric microparticles or implantable devices) may increase the efficiency of delivery.

The compositions can be formulated in various ways for parenteral or nonparenteral administration. Where suitable, oral formulations can take the form of tablets, pills, capsules, or powders, which may be enterically coated or otherwise protected. Sustained release formulations, suspensions, elixirs, aerosols, and the like can also be used.

Pharmaceutically acceptable carriers and excipients can be incorporated (e.g., water, saline, aqueous dextrose, and glycols, oils (including those of petroleum, animal, vegetable or synthetic origin), starch, cellulose, talc, glucose, lactose, sucrose, gelatin, malt, rice, flour, chalk, silica gel, magnesium stearate, sodium stearate, glycerol monosterate, sodium chloride, dried skim milk, glycerol, propylene glycol, ethanol, and the like). The compositions may be subjected to conventional pharmaceutical expedients such as sterilization and may contain conventional pharmaceutical additives such as preservatives, stabilizing agents, wetting or emulsifying agents, salts for adjusting osmotic pressure, buffers, and the like. Suitable pharmaceutical carriers and their formulations are described in “Remington's Pharmaceutical Sciences” by E. W. Martin, which is herein incorporated by reference. Such compositions will, in any event, contain an effective amount of the compositions together with a suitable amount of carrier so as to prepare the proper dosage form for proper administration to the patient.

The pharmaceutical compositions as disclosed herein can be prepared for oral or parenteral administration. Pharmaceutical compositions prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration. Aerosol inhalation can also be used. Thus, compositions can be prepared for parenteral administration that includes imipramine or any of the PARP inhibitors, PD-L1 inhibitors or PD-1 inhibitors dissolved or suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like).

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules.

In an aspect, a pharmaceutical composition comprises imipramine; and a) a PARP inhibitor, a PD-L1 inhibitor or a PD-1 inhibitor; and b) optionally, a pharmaceutical acceptable carrier. Further, the pharmaceutical composition comprises imipramine, the PARP inhibitor, the PD-L1 inhibitor and a PD-1 inhibitor in therapeutically effective amounts. In an aspect, the PARP inhibitor can be olaparib, or niraparib or veliparib or talazoparibolaparib. In an aspect, the PARP inhibitor is olaparib. In an aspect, the PD-L1 inhibitor can be anti-PD-L1 antibody. In an aspect, the anti-PD-L1 antibody can be selected from BMS-936559, durvalumab, atezolizumab or avelumab. In an aspect, the PD-1 inhibitor can be an anti-PD-1 antibody. In an aspect, the PARP inhibitor can be olaparib, the PD-L1 inhibitor can be an anti-PD-L1 antibody and the PD-1 inhibitor can be an anti-PD-1 antibody. In an aspect, the pharmaceutical composition can be formulated for oral or intravenous administration. In an aspect, the composition can be formulated in a lipid emulsion.

Articles of Manufacture

The composition described herein can be packaged in a suitable container labeled, for example, for use as a therapy to treat cancer or any of the methods disclosed herein. Accordingly, packaged products (e.g., sterile containers containing the composition described herein and packaged for storage, shipment, or sale at concentrated or ready-to-use concentrations) and kits, including at least imipramine as described herein and instructions for use, are also within the scope of the disclosure. A product can include a container (e.g., a vial, jar, bottle, bag, or the like) containing the composition described herein. In addition, an article of manufacture further may include, for example, packaging materials, instructions for use, syringes, buffers or other control reagents for treating or monitoring the condition for which prophylaxis or treatment is required. The product may also include a legend (e.g., a printed label or insert or other medium describing the product's use (e.g., an audio- or videotape)). The legend can be associated with the container (e.g., affixed to the container) and can describe the manner in which the compound therein should be administered (e.g., the frequency and route of administration), indications therefor, and other uses. The compounds can be ready for administration (e.g., present in dose-appropriate units), and may include a pharmaceutically acceptable adjuvant, carrier or other diluent. Alternatively, the compounds can be provided in a concentrated form with a diluent and instructions for dilution.

In an aspect, imipramine and the PARP inhibitor can be co-packaged. In an aspect, the imipramine, the PARP inhibitor and the PD-L1 inhibitor can be co-packaged. In an aspect, imipramine, the PARP inhibitor and the PD-1 inhibitor can be co-packaged. In an aspect, imipramine, the PARP inhibitor, the PD-L1 inhibitor and the PD-1 inhibitor can be co-packaged.

### EXAMPLES

### Example 1: Imipramine Inhibits the Growth of Breast Cancer Cells

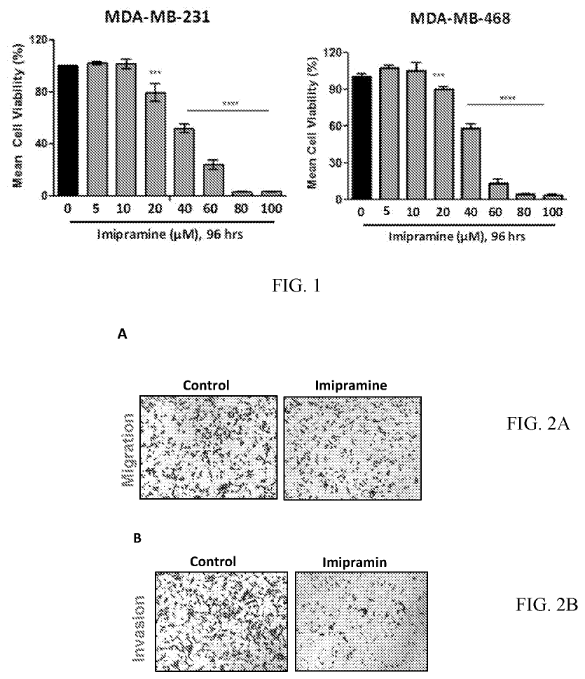

To test whether imipramine can inhibit breast cancer growth and progression, breast cancer cell lines (MDA-MB-231 and MDA-MB-468) were treated with vehicle as a control (DMSO) and an increasing dose of imipramine (5-100 μM) for 96 hours. Cell viability was assessed using alamar Blue cell viability assay. As shown in FIG. 1, imipramine treatment resulted in significantly reduced viability of breast cancer cells compared to vehicle-treated breast cancer cells.

### Example 2: Imipramine Inhibits Migration and Invasion of Breast Cancer Cell

To test whether imipramine may also target breast cancer progression, breast cancer cells were treated with imipramine and transwell migration and invasion assays were carried out. As shown in FIGS. 2A-B, imipramine treatment led to reduction in the migration as well as invasion of breast cancer cells when compared with vehicle control-treated cells.

### Example 3: Imipramine Inhibits Breast Cancer Growth In Vivo

To confirm imipramine's anti-tumor effect, an orthotopic xenograft model study was carried out. MDA-MB-231 cells were implanted into the mammary fat pad of athymic nude mice. After a week when tumors reached measurable size (e.g., approximately 100-200 mm³), mice were treated with imipramine or vehicle. Vehicle control or imipramine at two different concentrations: (1) 16 mg/kg body weight, equivalent to 100 mg dose for a 75 kg human escalated to 20 mg/kg body weight, equivalent to 125 mg/75 kg human body weight; and (2) 32 mg/kg body weight, equivalent to 200 mg dose for a 75 kg human escalated to 40 mg/kg body weight, equivalent to 250 mg/75 kg human body weight, were injected intra-peritoneally (ip) daily for thirty days. After the 30th day, the mice were euthanized, and the tumors were isolated and processed for molecular and immunohistologic studies. Tumor volume was calculated by using the formula 0.5236L₁ (L₂)², where L₁ is long axis and L₂ is the short axis of the tumor.

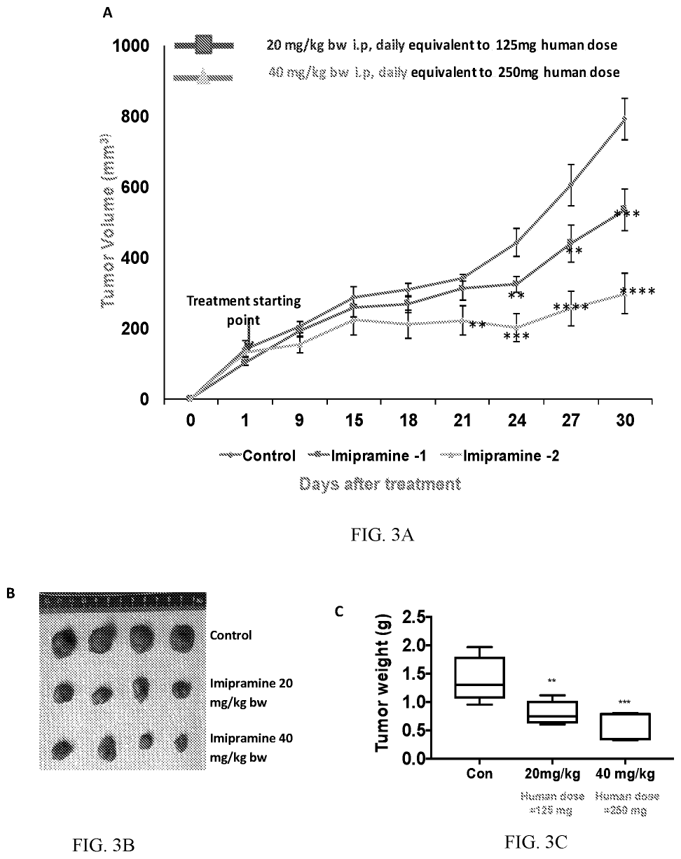

As shown in FIGS. 3A-C, imipramine treatment significantly reduced tumor growth (FIG. 3A), tumor size (FIG. 3B) and tumor weight (FIG. 3C) when compared to vehicle control.

### Example 4: Imipramine Improves Efficacy of Olaparib for Treating TNBC

To evaluate whether imipramine can sensitize the response to olaparib treatment, an orthotopic xenograft study was performed. MDA-MB-231-GFP-luc cells were implanted into the mammary fat pad of athymic nude mice and after a week when tumors reached measurable size (approximately 100-200 mm³), mice were treated with either (1) vehicle, (2) imipramine (40 mg/kg body weight equivalent to 250 mg human dose), (3) olaparib (50 mg/kg body weight=307 mg human dose), (4) imipramine+olaparib. Mice were treated for 4 weeks starting week 1 after tumor implantation. Tumor volumes were measured twice a week.

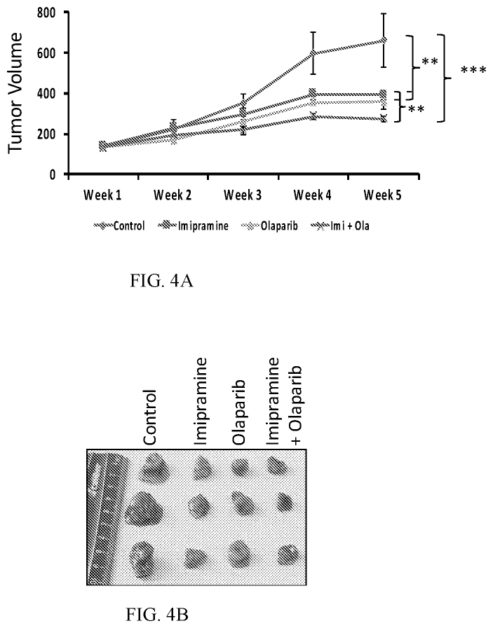

As shown in FIG. 4, the imipramine+olaparib combination treatment significantly reduced tumor growth when compared to vehicle (control). The human equivalent dose of olaparib used in this study is significantly lower that the approved dose for treating patients (400 mg/twice a day). A significant difference in tumor growth was observed starting at week 3 for imipramine vs the combination (imi+ola) and control vs imi+ola treatment, while for weeks 4 and 5 a significant difference was observed for all groups (control vs imipramine/olaparib; control vs imi+ola; imipramine vs imi+ola; and olaparib vs imi+ola).

### Example 5: Imipramine for Inhibiting Breast Cancer Brain Metastasis

Breast cancer is a common cause of brain metastases. TNBC and human epidermal growth factor receptor 2 (HER2)-positive breast cancer are reported to have an increased risk for the development of brain metastases. The potential of imipramine alone and in combination with olaparib and anti-PD-L1 and/or a PD-1 antibody to inhibit TNBC brain metastasis in a pre-clinical mouse model will be tested. These results will confirm that imipramine crosses the blood-brain barrier and can be a potent therapeutic regimen for treating breast cancer brain metastasis patients.

Tumor cell lines. Brainotropic TNBC MDA-MB-231_{Brain_met} cells that are aggressive and have a tendency to migrate to the brain were generated. Brainotropic MDA-MB-231_{Brain_met} cells were generated by selecting for MDA-MB-231 cells metastasized to the brain followed by another round of cardiac injection (for brain metastasis) using the selected cells. MDA-MB-231_{Brain_met} cells were finally selected after three rounds of brain metastasis. Since these cells express luc-GFP reporter, tumor growth in brain could be followed.

Xenograft model. For breast cancer brain metastasis, a cardiac injection model will be used. Single cell suspensions of MDA-MB-231_{Brain_met}-GFP-luc expressing cells will be used for cardiac injection in NSG (NOD/Scid/common gamma chain KO) mice. Model cells (1×10⁵) will be used for intracardiac injection. The following treatment schedule will be used: imipramine (40 mg/kg body weight=250 mg human dose, i.p daily) as monotherapy and imipramine (40 mg/kg body weight)+Olaparib (50 mg/kg body weight, i.p three times a week) or imipramine+anti-PD-L1 antibody (200 μg/mouse, four doses i.v,) will be initiated when the luciferase signal is detected in the brain. Imipramine and olaparib doses were chosen based on preliminary studies. The xenograft studies will contain the following treatment groups (n=10/group): Group I: DMSO (control); Group II: imipramine; Group III: olaparib; Group IV: imipramine+olaparib; Group V: anti-PD-L1 antibody; Group VI: imipramine+anti-PD-L1 antibody; Group VII: anti-PD-L1 antibody+olaparib; Group VIII: imipramine+anti-PD-L1 antibody+olaparib.

Outcome measures. The effect of imipramine and imipramine+anti-PD-L1 antibody and/or olaparib on TNBC brain metastasis will be measured by the Xenogen In Vivo Imaging System (Hopkinton, Mass.). Mice will be imaged weekly (from week 1-6) until they show morbidity. At the end of the desired treatment schedule, metastatic lesions will be isolated and processed for RNA for qRT-PCR analysis and for IHC analysis using antibodies against imipramine target genes as described herein.

Statistical analysis. Bio-statistical evaluation will be performed using a repeated-measures general linear modeling approach. The minimum sample size of 10 animals for each experimental group can be determined using Time-Averaged Difference Power Analysis to detect a 50 percent reduction in treated tumor cell volume with power>0.80% and α=0.05 (1-sided)(Liu et al., 2005). Drug combination will be calculated using multiple drug-effect equation, quantitated by the combination index (CI) where CI=1 indicates that the two drugs have additive effect, CI<1=“synergism” and CI>1=“antagonism”.

### Example 6: The Mechanism of the Imipramine Anti-Tumor Activities

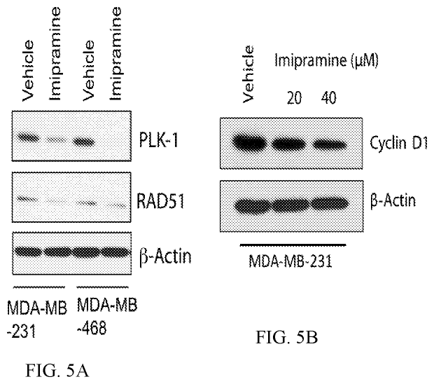

It was tested whether imipramine inhibits TNBC growth/progression by inhibiting cell cycle progression and DNA repair ability of TNBC cells. Supporting this, significantly reduced levels of cyclin D1, PLK1 and Rad51 were observed (FIG. 5). Cyclin D1, PLK1, and Rad51 are well-established regulators of cell cycle progression and DNA damage response (DDR). Furthermore, it is possible that imipramine may suppress immunosuppressive cytokines in a tumor microenvironment and therefore improve the efficacy of anti-PD-L1-based therapy. Supporting this notion, imipramine has been reported to inhibit IL-10 (Mukherjee et al., 2014), which has an immunosuppressive role in a tumor microenvironment. Importantly, imipramine is shown to elevate the levels of IL-12 (Mukherjee et al., 2014), which is proposed to be a candidate for tumor immunotherapy as it activates innate immunity via natural killer (NK) cells and adaptive immunity via cytotoxic T-lymphocytes (Lasek et al., 2014).

Described herein, these possibilities can be tested by determining the levels of DDR proteins in tumor tissues and also by determining the levels of cytokines in both tumor tissues and blood samples collected from imipramine treated patients. For an in-depth understanding of the imipramine effect on DDR, DNA fiber analysis in imipramine-treated TNBC cell lines and also in tumor tissues will be carried out.

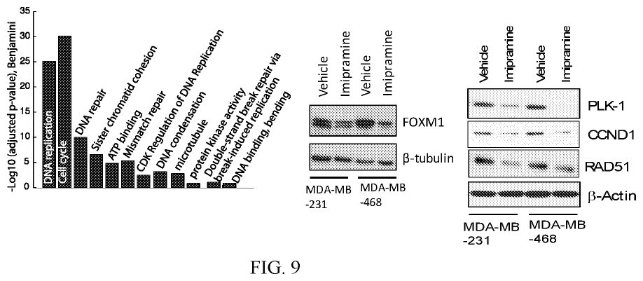

To understand the mechanism by which imipramine may inhibit TNBC growth and progression, the levels of genes that are known to play a role in cell cycle progression (see above) and also DNA repair was determined. Western blot (FIG. 5) analysis revealed that imipramine treatment resulted in significantly reduced levels of cyclin D1, PLK1 and Rad51. IPA analysis showed that DNA repair and cell cycle are two biological pathways that are highly enriched in imipramine treated TNBC cells (FIG. 9). Example of cell cycle and DNA repair genes that showed reduced expression in imipramine-treated TNBC cells included FOXM1, CyclinD1, PLK1, SKP2, XRCC3 and RAD51 (FIG. 9). The western bot analysis further validated the RNA-seq results as imipramine treatment significantly reduced FOXM1, and its targets, PLK1, CCND1 and RAD51 in TNBC cells. These imipramine target genes are known transcriptional targets of FOXM1 and are known to be highly expressed in TNBC patients.

RNA seq and western blot analysis: Total RNA was isolated from MDA-MB-231 cells following treatment with vehicle and Imipramine for 96 hours, respectively. RNA samples were further processed for gene expression profiling using Illumina HiSeq 2000 following manufacturer's standard protocol (Illumina, San Diego, Calif.). Differential expression analysis was performed by using DEseq, and significant genes with at least 1.5-fold change with p<0.05 were chosen for analysis. Using all significant and differentially expressed genes from the RNA-seq data, Ingenuity Pathway Analysis software (IPA) was used to interpret the biological pathways. Total protein extracted from cell lines were subjected to western blot analysis.

Measuring the cytokine levels in blood and tissue samples from imipramine-treated patients. It is possible that imipramine will inhibit immunosuppressive cytokines and elevate cytokines that activate NK and killer T-cells in tumor microenvironment. To test this, cytokine levels will be measured in serum and tumor tissues from imipramine-treated TNBC patients, a human 17-plex panel will be used that includes IL-10 and IL-12 in addition to other cytokines and standardized ELISA kits. For serum profiling of interleukins, peripheral blood will be obtained at baseline, 2 days, 7 days, 14 days, 21 days and 28 days following the first dose of imipramine. Blood will be collected in a heparin-containing vacutainer, spun down, plasma aliquoted and will be frozen immediately. For tumor tissue, tumor supernatant will be prepared by lysing the tissues in lysis buffer followed by orbital agitation and centrifugation. The tissue supernatant will be collected and used for cytokine assay. Cytokine assay will be done in all imipramine treated patients including those patients that will be treated with imipramine combination therapy.

Correlate changes in molecular markers of imipramine with tumor response. Imipramine target gene levels and DDR response in tumor tissues will be evaluated. Surgical specimens from control and imipramine-treated patients will be processed for formalin fixation/paraffin-embedding and for ex-vivo explants. Formalin-fixed samples will be subjected to immunohistochemical staining for protein markers including cyclin D1, PLK1 and Rad51 that showed significant changes in our pre-clinical tumor tissues. Diaminobenzidine-streptavidin technique with microwaving for antigen retrieval will be used. Subsequent steps will be performed using automated equipment. All primary antibodies will be applied overnight at 4° C. After washing with phosphate-buffered saline (PBS), sections will be incubated with appropriate secondary antibodies, antirabbit or antimouse at 1:2,000 (Jackson ImmunoResearch Laboratories, West Grove, Pa.), for 30 minutes and washed in PBS. Vectastain Elite ABC reagent (Vector Laboratories, Burlingame, Calif.) will be applied for 30 minutes, and sections will be washed in PBS. Finally, the color will be developed by incubation with diaminobenzidine solution (Vector Laboratories), and sections will be counterstained with hematoxylin. For scoring, traditional semiquantitative analysis will be performed by a trained pathologist utilizing a cumulative scoring system of a 1-3 score for intensity and a 1-3 score for percentage of cells staining. Additionally, semiautomated image analysis and semiquantitative scoring will be performed using Image ProPlus Software (Media Cybernetics, Inc., Bethesda, Md.).

To test if imipramine suppresses TNBC growth and metastasis as well as sensitizes PARP inhibitor response by affecting cell cycle progression and DNA damage response. The studies described herein suggest that imipramine may inhibit TNBC growth and progression as well as sensitize PARP inhibitor, Olaparib, response by altering DNA repair ability of TNBC cells (FIG. 5). In vitro and cells isolated from tumor tissues will be used to examine whether suppression of DNA repair ability may be one of the mechanisms by which imipramine inhibits TNBC cell growth.

The studies disclosed herein show that imipramine treatment results in significantly reduced levels of RAD51, which is known to play an important role in homologous recombination (HR)-mediated DNA repair (Baumann and West, 1998). To further confirm that imipramine indeed affects DNA repair ability of TNBC cells, a functional assay to monitor HR events will be performed. The ISceI-based DR-GFP reporter assay that measures the frequency of double strand break repair by HR (Gunn et al., 2011, Stark, 2004; Rajamanickam et al., 2016; Stark et al., 2004) will be used. TNBC cells stably expressing DR-GFP reporter will be treated with or without imipramine followed by FACS analysis. A significantly reduced number of GFP positive cells in imipramine-treated cells will support the notion that imipramine indeed inhibits HR.

### Example 7: To Test the Safety and Efficacy of Imipramine as a Potential Therapeutic Regimen for Treating Breast Cancers in Clinical Trials

The results described herein demonstrate that imipramine inhibits TNBC growth and invasion (FIGS. 1, 2, 3). While the results showing that imipramine's anti-tumor activity is significant, a clinical study will be carried out to establish the therapeutic efficacy of imipramine for treating TNBC patients. Three parallel phase I clinical trials will be performed to evaluate the safety and efficacy of imipramine alone or in in combination with niraparib and anti-PD-1.

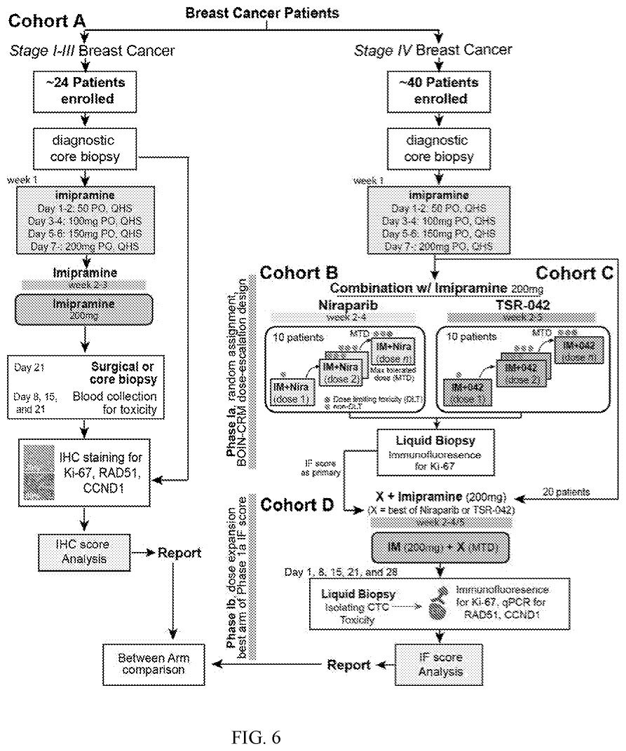

Experimental Design. Three parallel single center clinical trials will be carried out using imipramine alone and in combination with PARP inhibitor (e.g., niraparib) and TSR-042 antibody (see, FIG. 6) and a dose expansion of the combination with the most promising preliminary efficacy data. In all cohorts, there will be a one week dose escalation of imipramine to a dose of 200 mg PO daily. The two phase I cohorts will be run in the BOIN model which has been shown to be more efficient than the 3+3 design (Yuan et al., 2016). The primary endpoint of cohort A will be changes in Ki67 between pre- and post-treatment specimens. Secondary endpoints include safety, changes in total cyclin D1 and RAD51 expression. The primary endpoint of Cohorts B and C will be safety with secondary endpoints being response rate, absolute change in the Ki67, pharmacokinetic (PK) analysis and assessment of total cyclin D1 and RAD51 expression. These trials will roll into cohort D, a phase Ib dose expansion trial based on the preliminary results for efficacy of each combination. The primary endpoint of cohort D is absolute change in the Ki67 in CTCs with secondary endpoints response rate, safety and changes in total cyclin D1 and RAD51 expression.

TABLE 1
The main participation criteria that will be used.

| | | |
|---|---|---|
| Inclusion | 1. | Previously untreated stage I-III breast cancer determined by a core needle biopsy |
| Criteria | | showing invasive ductal carcinoma or invasive lobular carcinoma. |
| Cohort A | 2. | Estrogen and progesterone receptor negative - defined as less than 1% staining by IHC. |
| | 3. | HER2 negative defined as 0 or 1+ using IHC or a ratio of less than 2.0 on FISH |
| | | testing. HER2 of 2+ on IHC should have a ratio of less than 2.0 on FISH testing to |
| | | be considered HER2 negative. |
| | 4. | Females of childbearing potential must have a negative serum or urine beta human |
| | | chorionic gonadotropin (β-hCG) pregnancy test result within 14 days prior to the |
| | | first dose of imipramine. |
| | 5. | Patients must be eligible for surgical resection of their breast cancer or repeat |
| | | biopsy after completing treatment. |
| | 6. | Patients must have a performance status of ECOG 0, 1. |
| | 7. | Tissue block of initial biopsy specimen is available. |
| Exclusion | 1. | Known diagnosis of bipolar depression or psychosis |
| criteria | 2. | Age >=70 years |
| Cohort A | 3. | Renal impairment defined as EGFR <30 |
| | 4. | Hepatic impairment as judged by clinical investigator or bilirubin >2 |
| | 5. | As judged by the investigator, severe uncontrolled concurrent medical |
| | | conditions, psychiatric illness or social condition that would limit |
| | | compliance with study requirements |
| | 6. | History of cardiac disease (arrhythmia, conduction abnormality, congenital |
| | | prolonged QT syndrome, or prolonged QTc rhythm noted during initial EKG >480 ms) |
| | 7. | Current use of SSRI, SNRI, MAO inhibitor, tramadol or trazadone; or use of these |
| | | agents within 14 days |
| | 8. | Inflammatory breast cancer |
| | 9. | Suicidal ideation or history of suicide attempt |
| | 10. | Myocardial infarction within 3 months of study initiation. |
| | 11. | Patients with Angle-Closure Glaucoma |
| | 12. | Pregnant or breast-feeding women. |
| Inclusion | 1. | Stage IV breast cancer determined by a core needle biopsy showing invasive ductal |
| Criteria | | carcinoma or invasive lobular carcinoma. |
| Cohorts | 2. | Estrogen and progesterone receptor negative - defined as less than 1% staining by IHC. |
| B, C, D | 3. | HER2 negative defined as 0 or 1+ using IHC or a ratio of less than 2.0 on FISH |
| | | testing. HER2 of 2+ on IHC should have a ratio of less than 2.0 on FISH testing to |
| | | be considered HER2 negative. |
| | 4. | Patients must have a performance status of ECOG 0, 1, 2. |
| | 5. | Tissue block of initial biopsy specimen or metastatic disease is available. |
| Exclusion | 1. | Known diagnosis of major depressive disorder, bipolar depression or psychosis |
| Criteria | 2. | ECOG 3 or 4 |
| Cohorts | 3. | Age >=70 years |
| B, C, D: | 4. | Renal impairment defined as EGFR <30 |
| | 5. | Hepatic impairment as judged by clinical investigator or bilirubin >2 |
| | 6. | As judged by the investigator, severe uncontrolled concurrent medical |
| | | conditions, psychiatric illness or social condition that would limit |
| | | compliance with study requirements |
| | 7. | History of cardiac disease (arrhythmia, conduction abnormality, congenital |
| | | prolonged QT syndrome, or prolonged QTc rhythm noted during initial EKG >480 ms) |
| | 8. | Current use of SSRI, SNRI, MAO inhibitor, tramadol or trazadone; or use of |
| | | these agents within 14 days |
| | 9. | Suicidal ideation or history of suicide attempt |
| | 10. | Myocardial infarction within 3 months of study initiation. |
| | 11. | Patients with Angle-Closure Glaucoma |
| | 12. | Pregnant or breast-feeding women. |
| Additional | 1. | Active autoimmune disease that has required systemic treatment for the |
| Exclusion | | past 2 years |
| criteria for | 2. | Use of immunosuppressive agents (corticosteroids, disease modifying agents). |
| Cohort C: | | Physiologic corticosteroid therapy is allowed. |
| | 3. | Prior allogeneic or solid organ transplant |
| | 4. | History of non-infectious pneumonitis that required steroids |
| | 5. | Known history of Human Immunodeficiency Virus (HIV), active Hapatitis B or |
| | | Hepatitis C |
| | 6. | Has received a live vaccine within 30 days of planned initiation of therapy |

### Subjects, Treatment Plan, and Endpoint.

Cohort A—Imipramine Window Trial. This study will include up to total of 24 experimental subjects. After having a core needle biopsy of the breast confirming triple negative breast cancer, eligible patients enrolled in the study will be treated with imipramine at a target dose of 200 mg PO daily for about 21 days (range 21-30 days) (for example, see, FIG. 6). During this treatment period, patients will continue with routine pre-operative planning and evaluations. This study will not delay patients proceeding to surgical intervention. Patients will continue on imipramine until the day prior to surgery and will stop taking imipramine after the evening dose the day before surgery. Since the time from diagnosis to surgery varies slightly between patients, the aim will be for 21 days of treatment, but will include women who receive treatment for up to 30 days. This ensures that there is not a delay in proceeding with standard of care treatment for early stage breast cancer which is surgical resection. It also ensures that imipramine will not be stopped several days prior to surgical resection. Patients will be evaluated on day 8, day 15, day 21 and at the end of treatment for toxicity. If patients are not expected to have surgery, such as those requiring neoadjuvant chemotherapy, a repeat core needle biopsy will be conducted in the medical oncology clinic on day 21 (+7 days). Patients will then be re-evaluated in the medical oncology clinic on day 42 visit (+/−7 days) as part of a routine post-surgical or treatment follow up appointment with repeat physical exam, toxicity check and routine laboratory testing to evaluate for any toxicity to imipramine. The primary endpoint for this study will be the absolute change in the Ki67. Secondary endpoints will include assessment of total cyclin D1 and RAD51 expression by immunohisto-chemistry.

Dose Selection for Imipramine. Patients will begin treatment at 50 mg PO QHS (every night at bedtime) and then increase by 50 mg every other day to target dose of 200 mg QHS. If a patient experiences a significant drug related toxicity, defined as grade 3 or 4 based on the CTCAE version 4.03 criteria, then the drug will be stopped. This dose has been selected based on preclinical work showing efficacy of imipramine alone or in combination with PARP inhibitor (FIGS. 3 and 4). Furthermore, a dose of 100-300 mg daily can be used in clinical practice with solid short- and long-term safety data.

Cohorts B and C. These cohorts will include up to nine experimental subjects each with an accrual duration of 21 months. As shown in FIG. 5, after core biopsy confirmation of stage IV TNBC, eligible patients enrolled in the study will be treated with escalating imipramine from 50 mg to at a target dose of 200 mg PO daily for first 7 days. After the dose escalation period, a continual reassessment method (CRM) based on Bayesian Optimal Interval (BOIN) will be used, where, given a target level of toxicity and increased dose levels, and initial expectations of the probability of dose-limiting toxicity (DLT) at each dose will be constructed using a statistical dose-toxicity model. Briefly, three patients will be randomly selected and treated for an initial dose level of either niraparib for 3 weeks or TSR-042 for 4 weeks, combining with 200 mg imipramine. Blood samples will be drawn from patients on day 8, day 15, day 21 and at end of treatment for toxicity and efficacy analysis. Patients will be evaluated for toxicity as a primary end point and PK, absolute levels of Ki67 and expression levels of cyclin D1 and RAD51 as secondary end points using liquid biopsy. Biomarkers of response will be evaluated in circulating tumor cells isolated from patient blood. Patients will have imaging studies every two treatment cycles for assessment of clinical response. If the observed toxicity rate in 3 patients is less than a lower bound determined by the model (or no patient experiences toxicity), corresponding dose will be escalated for the next 3 patient cohort. With treatment of successive patient cohorts, the statistical model will be recalculated to update estimated probability of a DLT and increase certainty associated with dose-toxicity relationship. If a toxicity rate is greater than an upper bound determined by the model, the trial will stop. Dose associated with the target DLT rate according to the final dose-toxicity model at trial completion is defined as the maximum tolerated toxicity (MTD).

Cohort D: Dose-expansion. This cohort will include up to 20 experimental subjects with an accrual interval of 18 months after the completion of Cohorts B and C and the determination of best outcome of cohorts B and C. Similar to previous cohorts, eligible patients enrolled on the study will be treated with escalating imipramine from 50 mg to at a target dose of 200 mg PO daily for first 7 days. After the first week treatment, patients will continue to the combination treatment with imipramine and the selected drug X (either niraparib or TSR-042) at MTD. The primary endpoint for this study will include absolute change in the Ki67 by immunofluorescence (IF) of circulating tumor cells (CTC). Secondary endpoints will include the response rate and assessment of total cyclin D1 and RAD51 expression by qPCR.

### Example 8: Imipramine Inhibits PD-L1 Expression in TNBC

Meta-analysis of ENCODE data set revealed (Birney et al., Nature, 2012, 489 (7414): 49-51) that FOXM1 may bind to cis-acting elements of PD-L1. To further substantiate these findings, it was tested whether FOXM1 may transactivate PD-L1 expression. MDA-MB-231 cells were treated with different doses of imipramine (10, 20, 40 μM) and/or transfected with scramble siRNA and siFOXM1. Then cells were subjected to FITC-anti-PDL1 antibody incubation for 1 hr. Cell surface PD-L1 expression was analyzed using flow cytometry.

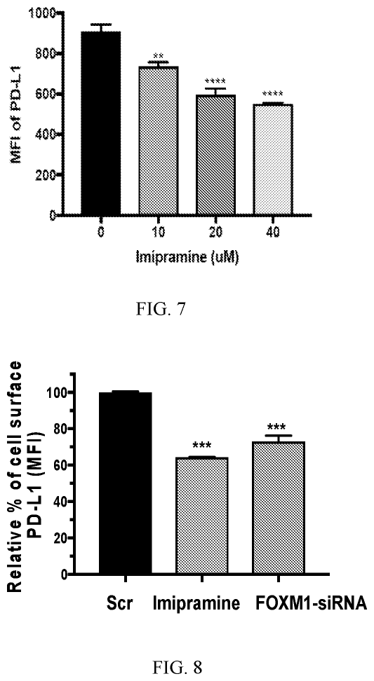

Indeed, silencing of FOXM1 reduced the levels of cell surface PD-L1 in TNBC cells as revealed by flow cytometric analysis (FIGS. 7 and 8). Imipramine treatment led to inhibition of cell surface PD-L1 expression (FIGS. 7 and 8) in TNBC cells. These results show that FOXM1 regulates PD-L1 expression and imipramine may inhibit TNBC growth and progression by inhibiting PD-L1 expression.

What is claimed is:

1. A method of treating cancer in a subject, the method comprising:
(a) identifying a subject in need of treatment; and
(b) administering to the subject a therapeutically effective amount of imipramine.

2. The method of claim 1, the subject has been diagnosed with cancer prior to the administering step.

3. The method of claim 1, wherein the subject is a human.

4. The method of claim 1, wherein the cancer is a primary or secondary tumor.

5. The method of claim 4, wherein the primary or secondary tumor is within the subject's breast, brain, lung or liver.

6. The method of claim 1, wherein the cancer is breast cancer, lung cancer, brain cancer or liver cancer.

7. The method of claim 1, wherein the cancer is triple negative breast cancer.

8. The method of claim 1, wherein imipramine is administered orally or parentally.

9. The method of claim 8, wherein the parental administration is intravenous, subcutaneous, intramuscular or direct injection.

10. The method of claim 1, wherein the therapeutically effective amount of imipramine is 75 mg to 300 mg/day.

11. The method of claim 1, further comprising administering a therapeutically effective amount of a PARP inhibitor.

12. The method of claim 11, wherein the PARP inhibitor is olaparib, or niraparib or veliparib or talazoparib.

13. The method of claim 11, wherein the PARP inhibitor is olaparib.

14. The method of claim 13, wherein administration of imipramine increases the efficacy of olaparib.

15. The method of claim 1, further comprising administering a therapeutically effective amount of a PD-L1 inhibitor.

16. The method of claim 15, wherein the PD-L1 inhibitor is an anti-PD-L1 antibody.

17. The method of claim 16, wherein the anti-PD-L1 antibody is BMS-936559, durvalumab, atezolizumab or avelumab

18. The method of claim 1, further comprising administering a therapeutically effective amount of a PD-1 inhibitor.

19. The method of claim 18, wherein the PD-1 inhibitor is an anti-PD-1 antibody;
wherein the anti-PD-1 antibody is nivolumab or pembrolizumab

20. The method of claim 1, further comprising administering a therapeutically effective amount of a PD-L1 inhibitor and a PD-1 inhibitor.

21. The method of claim 1, further comprising administering a therapeutically effective amount of a PARP inhibitor, a PD-L1 inhibitor and a PD-1 inhibitor.

22. The method of claim 21, wherein the PARP inhibitor is olaparib, the PD-L1 inhibitor is an anti-PD-L1 antibody and the PD-1 inhibitor is an anti-PD-1 antibody.

23. A method of treating cancer in a subject, the method comprising:
(a) identifying a subject in need of treatment; and
(b) administering to the subject a therapeutically effective amount of imipramine and a PARP inhibitor.

24. The method of claim 23, further comprising administering a therapeutically effective amount of PD-L1 inhibitor or a PD-1 inhibitor.

25. The method of claim 23, wherein imipramine and the PARP inhibitor are co-formulated.

26. The method claim 24, wherein imipramine, the PARP inhibitor and the PD-L1 inhibitor are co-formulated.

27. The method claim 24, wherein imipramine, the PARP inhibitor and the PD-1 inhibitor are co-formulated.

28. The method claim 24, wherein imipramine, the PARP inhibitor, the PD-L1 inhibitor and the PD-1 inhibitor are co-formulated.

29. The method of claim 23, wherein imipramine and the PARP inhibitor are co-packaged.

30. The method claim 24, wherein imipramine, the PARP inhibitor and the PD-L1 inhibitor are co-packaged.

31. The method claim 24, wherein imipramine, the PARP inhibitor and the PD-1 inhibitor are co-packaged.

32. The method claim 24, wherein imipramine, the PARP inhibitor, the PD-L1 inhibitor and the PD-1 inhibitor are co-packaged.

33. A method of inhibiting cell cycle progression, cell growth or DNA repair, the method comprising: contacting a cell or tissue or administering to a subject in need thereof, a therapeutically effective amount of imipramine.

34. The method of claim 33, further comprising administering a therapeutically effective amount of a PARP inhibitor.

35. The method of claim 34, wherein the PARP inhibitor is olaparib, or niraparib or veliparib or talazoparib.

36. The method of claim 34, wherein the PARP inhibitor is olaparib.

37. The method of claim 36, wherein administration of imipramine increases the efficacy of olaparib.

38. The method of claim 33, further comprising administering a therapeutically effective amount of a PD-L1 inhibitor.

39. The method of claim 38, wherein the PD-L1 inhibitor is an anti-PD-L1 antibody.

40. The method of claim 39, wherein the anti-PD-L1 antibody is BMS-936559, durvalumab, atezolizumab or avelumab.

41. The method of claim 33, further comprising administering a therapeutically effective amount of a PD-1 inhibitor.

42. The method of claim 41, wherein the PD-1 inhibitor is an anti-PD-1 antibody.

43. The method of claim 42, wherein the anti-PD-1 antibody is nivolumab or pembrolizumab or TSR-042.

44. The method of claim 33, further comprising administering a therapeutically effective amount of a PARP inhibitor, a PD-L1 inhibitor or a PD-1 inhibitor.

45. The method of claim 44, wherein the PARP inhibitor is olaparib, or niraparib or veliparib or talazoparib and the PD-L1 inhibitor is an anti-PD-L1 antibody and the PD-1 inhibitor is an anti-PD-1 antibody.

46. The method of claim 33, the subject has been diagnosed with cancer prior to the administering step.

47. The method of claim 46, wherein the cancer is a primary or secondary tumor or metastatic tumor.

48. The method of claim 47, wherein primary is within the subject's breast, brain, lung or liver.

49. The method of claim 46, wherein the cancer is triple negative breast cancer.

50. The method of claim 46, wherein the cancer is breast cancer, lung cancer, brain cancer or liver cancer.

51. The method of claim 33, wherein cell cycle progression, cell growth or DNA repair is inhibiting by inhibition of genes cyclin D1, PLK1 or Rad51.

52. A method of inhibiting growth, transformation or metastasis of cancer cells, the method comprising: contacting a cell or tissue or administering to a subject in need thereof, a therapeutically effective amount of imipramine.

53. The method of claim 52, further comprising administering a therapeutically effective amount of a PARP inhibitor.

54. The method of claim 53, wherein the PARP inhibitor is olaparib, or niraparib or veliparib or talazoparib.

55. The method of claim 53, wherein the PARP inhibitor is olaparib.

56. The method of claim 55, wherein administration of imipramine increases the efficacy of olaparib.

57. The method of claim 52, further comprising administering a therapeutically effective amount of a PD-L1 inhibitor.

58. The method of claim 57, wherein the PD-L1 inhibitor is an anti-PD-L1 antibody.

59. The method of claim 58, wherein the anti-PD-L1 antibody is BMS-936559, durvalumab, atezolizumab or avelumab.

60. The method of claim 52, further comprising administering a therapeutically effective amount of a PD-1 inhibitor.

61. The method of claim 60, wherein the PD-1 inhibitor is an anti-PD-1 antibody.

62. The method of claim 52, further comprising administering a therapeutically effective amount of a PARP inhibitor, a PD-L1 inhibitor and a PD-1 inhibitor.

63. The method of claim 62, wherein the PARP inhibitor is olaparib, the PD-L1 inhibitor is an anti-PD-L1 antibody and the PD-1 inhibitor is an anti-PD-1 antibody.

64. The method of claim 52, the subject has been diagnosed with cancer prior to the administering step.

65. The method of claim 64, wherein the cancer is a primary or secondary tumor.

66. The method of claim 65, wherein primary is within the subject's breast, brain, lung or liver.

67. The method of claim 64, wherein the cancer is triple negative breast cancer.

68. The method of claim 64, wherein the cancer is breast cancer, lung cancer, brain cancer or liver cancer.

69. A pharmaceutical composition comprising:
imipramine; and
a) a PARP inhibitor, a PD-L1 inhibitor or a PD-1 inhibitor; and
b) optionally, a pharmaceutical acceptable carrier;
wherein imipramine, the PARP inhibitor, the PD-L1 inhibitor and a PD-1 inhibitor are present in a therapeutically effective amount.

70. The composition of claim 69, wherein the PARP inhibitor is olaparib, or niraparib or veliparib or talazoparib.

71. The composition of claim 69, wherein the PARP inhibitor is olaparib.

72. The composition of claim 69, wherein the PD-L1 inhibitor is an anti-PD-L1 antibody.

73. The composition of claim 72, wherein the anti-PD-L1 antibody is selected from BMS-936559, durvalumab, atezolizumab or avelumab.

74. The composition of claim 69, wherein the PD-1 inhibitor is anti-PD-1 antibody.

75. The composition of claim 69, wherein the PARP inhibitor is olaparib, the PD-L1 inhibitor is an anti-PD-L1 antibody and the PD-1 inhibitor is an anti-PD-1 antibody.

76. The composition of claim 69, wherein the composition is formulated for oral or intravenous administration.

77. The composition of claim 69, wherein the composition is formulated in a lipid emulsion.
